(19) United States
(12) Patent Application Publication
(10) Pub. No.: US 2021/0322542 A1
(43) Pub. Date: Oct. 21, 2021

(54) VACCINATION AGAINST CORONAVIRUS WITH POLIOMYELITIS VACCINE

(71) Applicant: Qiyi Xie, San Diego, CA (US)

(72) Inventor: Qiyi Xie, San Diego, CA (US)

(21) Appl. No.: 17/098,449

(22) Filed: Nov. 15, 2020

Related U.S. Application Data
(60) Provisional application No. 63/013,561, filed on Apr. 22, 2020.
(60) Provisional application No. 63/010,678, filed on Apr. 15, 2020.
(60) Provisional application No. 63/008,664, filed on Apr. 11, 2020.

(51) Int. Cl. A61K 39/295; A61P 31/14

(52) U.S. Cl. CPC A61K 39/295; A61K 2039/58; A61P 31/14

(57) ABSTRACT

Provided herein is a method for preventing an infectious disease caused by a Coronaviridae virus with a poliomyelitis vaccine. Also provided herein is a method of inducing a protective immune response against a Coronaviridae virus with a poliomyelitis vaccine.

### CROSS REFERENCE TO RELATED APPLICATIONS

This application claims the benefit of the priority of U.S. Provisional Application Nos. 63/008,664, filed Apr. 11, 2020; 63/010,678, filed Apr. 15, 2020; and 63/013,561, filed Apr. 22, 2020; the disclosure of each of which is incorporated herein by reference in its entirety.

### FIELD

Provided herein is a method for preventing an infectious disease caused by a Coronaviridae virus with a poliomyelitis vaccine. Also provided herein is a method of inducing a protective immune response against a Coronaviridae virus with a poliomyelitis vaccine.

### REFERENCE TO A SEQUENCE LISTING

The present specification is being filed with a Sequence Listing in Computer Readable Form (CRF), which is entitled 805A002US01_SEQ_LISTING_ST25.txt of 8,322 bytes in size and created Nov. 15, 2020; the content of which is incorporated herein by reference in its entirety.

### BACKGROUND

Coronavirus disease 2019 (COVID-19) is caused by severe acute respiratory syndrome coronavirus-2 (SARS-CoV-2). Gorbalenya et al., Nat. Microbiol. 2020, 5, 536-44; Zhang et al., Science 2020, 368, 409-12. On Mar. 11, 2020, the World Health Organization declared COVID-19 a global pandemic. Zhang et al., Science 2020, 368, 409-12; Dai et al., Science 2020, 368, 1331-5. By mid-July of 2020, COVID-19 has spread almost to every corner of the world. As of October 2020, there are almost 50 million confirmed cases and over a million confirmed deaths globally. The COVID-19 pandemic poses as a great public health threat to the world.

COVID-19 can be fatal, especially to older people and those with pre-existing medical conditions. Shahid et al., J. Am. Geriatr. Soc. 2020, 68, 926-9. Common symptoms of COVID-19 include fever or chills, cough, shortness of breath or difficulty breathing, fatigue, muscle or body aches, headache, loss of taste or smell, sore throat, congestion or runny nose, nausea or vomiting, and diarrhea. Id. Currently, there is no FDA-approved vaccine for COVID-19. Therefore, there is an urgent need for an immunization method to battle the COVID-19 pandemic.

### SUMMARY OF THE DISCLOSURE

Provided herein is a method for preventing a person from being infected by a Group IV virus, comprising administrating to the person an effective amount of a poliomyelitis vaccine.

Also provided herein is a method for treating a person with an infectious disease caused by a Group IV virus, comprising administrating to the person an effective amount of a poliomyelitis vaccine.

Additionally, provided herein is a method of inducing a protective immune response against a Coronaviridae virus in a person, comprising administering to the person an immunologically effective amount of a poliomyelitis vaccine.

Furthermore, provided herein is a method of inducing a protective gastrointestinal immune response against a Coronaviridae virus in a person, comprising administering to the person an immunologically effective amount of a poliomyelitis vaccine.

Provided herein is a method of inducing an immune response protective against severe acute respiratory syndrome caused by an infection of a Coronaviridae virus in a person, comprising administering to the person an immunologically effective amount of a poliomyelitis vaccine.

Provided herein is a method of reducing the severity of one or more symptoms of an infectious disease caused by a Coronaviridae virus in a person, comprising administering to the person an immunologically effective amount of a poliomyelitis vaccine.

Provided herein is a method of reducing the severity of one or more symptoms of severe acute respiratory syndrome caused by an infection of a Coronaviridae virus in a person, comprising administering to the person an immunologically effective amount of a poliomyelitis vaccine.

Provided herein is a method of inhibiting replication of a Coronaviridae virus in a person, comprising administering to the person an immunologically effective amount of a poliomyelitis vaccine.

### DETAILED DESCRIPTION

To facilitate understanding of the disclosure set forth herein, a number of terms are defined below.

Generally, the nomenclature used herein and the laboratory procedures in biochemistry, biology, immunology, virology, and pharmacology described herein are those well-known and commonly employed in the art. Unless defined otherwise, all technical and scientific terms used herein generally have the same meaning as commonly understood by one of ordinary skill in the art to which this disclosure belongs.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a person from acquiring a disorder, disease, or condition; or reducing a person's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a person derives from a prophylactic or therapeutic agent do not result in a cure of the disorder, disease, or condition.

The term “effective amount” or “immunologically effective amount” is meant to include the amount of a vaccine that, when administered, is sufficient to prevent development of, or alleviate to some extent, one or more of the symptoms of the disorder, disease, or condition being prevented or alleviated.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

In one embodiment, provided herein is a method for preventing a person from being infected by a Group IV virus, comprising administering to the person an effective amount of a poliomyelitis vaccine.

In another embodiment, provided herein is a method for treating a person with an infectious disease caused by a Group IV virus, comprising administering to the person an effective amount of a poliomyelitis vaccine.

In certain embodiments, the Group IV virus is an Arteriviridae, Astroviridae, Caliciviridae, Coronaviridae, Flaviviridae, Picornaviridae, or Togaviridae virus. In certain embodiments, the Group IV virus is an Arteriviridae, virus. In certain embodiments, the Group IV virus is an Astroviridae virus. In certain embodiments, the Group IV virus is a Caliciviridae virus. In certain embodiments, the Group IV virus is a Coronaviridae virus. In certain embodiments, the Group IV virus is a Flaviviridae virus. In certain embodiments, the Group IV virus is a Picornaviridae virus. In certain embodiments, the Group IV virus is a Togaviridae virus.

In certain embodiments, the Group IV virus is an arterivirus, astrovirus, calicivirus, coronavirus, flavivirus, picornavirus, or togavirus. In certain embodiments, the Group IV virus is an arterivirus. In certain embodiments, the Group IV virus is an astrovirus. In certain embodiments, the Group IV virus is a calicivirus. In certain embodiments, the Group IV virus is a coronavirus. In certain embodiments, the Group IV virus is a SARS coronavirus. In certain embodiments, the Group IV virus is a COVID-19 coronavirus. In certain embodiments, the Group IV virus is a flavivirus. In certain embodiments, the Group IV virus is a picornavirus. In certain embodiments, the Group IV virus is a togavirus.

In certain embodiments, the Group IV virus is a Betacoronavirus. In certain embodiments, the Group IV virus is a Coronavirinae virus. In certain embodiments, the Group IV virus is a severe acute respiratory syndrome coronavirus-1 (SARS-CoV-1). In certain embodiments, the Group IV virus is a severe acute respiratory syndrome coronavirus-2 (SARS-CoV-2). In certain embodiments, the Group IV virus is a Middle East respiratory syndrome-related coronavirus (MERS-CoV).

In certain embodiments, the infectious disease is caused by an Arteriviridae, Astroviridae, Caliciviridae, Coronaviridae, Flaviviridae, Picornaviridae, or Togaviridae virus. In certain embodiments, the infectious disease is caused by an Arteriviridae, virus. In certain embodiments, the infectious disease is caused by an Astroviridae virus. In certain embodiments, the infectious disease is caused by a Caliciviridae virus. In certain embodiments, the infectious disease is caused by a Coronaviridae virus. In certain embodiments, the infectious disease is caused by a Flaviviridae virus. In certain embodiments, the infectious disease is caused by a Picornaviridae virus. In certain embodiments, the infectious disease is caused by a Togaviridae virus.

In certain embodiments, the infectious disease is caused by an arterivirus, astrovirus, calicivirus, coronavirus, flavivirus, picornavirus, or togavirus. In certain embodiments, the infectious disease is caused by an arterivirus. In certain embodiments, the infectious disease is caused by an astrovirus. In certain embodiments, the infectious disease is caused by a calicivirus. In certain embodiments, the infectious disease is caused by a coronavirus. In certain embodiments, the infectious disease is caused by a SARS coronavirus. In certain embodiments, the infectious disease is caused by a COVID-19 coronavirus. In certain embodiments, the infectious disease is caused by a flavivirus. In certain embodiments, the infectious disease is caused by a picornavirus. In certain embodiments, the infectious disease is caused by a togavirus.

In certain embodiments, the infectious disease is caused by a Betacoronavirus. In certain embodiments, the infectious disease is caused by a Coronavirinae virus. In certain embodiments, the infectious disease is caused by a SARS-CoV-1. In certain embodiments, the infectious disease is caused by a SARS-CoV-2. In certain embodiments, the infectious disease is caused by a MERS-CoV.

In certain embodiments, the infections disease is coronavirus disease, which is also known as COVID-19. In certain embodiments, the infectious disease is severe acute respiratory syndrome.

In yet another embodiment, provided herein is a method of inducing a protective immune response against a Coronaviridae virus in a person, comprising administering to the person an immunologically effective amount of a poliomyelitis vaccine.

In yet another embodiment, provided herein is a method of inducing a protective gastrointestinal immune response against a Coronaviridae virus in a person, comprising administering to the person an immunologically effective amount of a poliomyelitis vaccine.

In yet another embodiment, provided herein is a method of inducing an immune response protective against severe acute respiratory syndrome caused by an infection of a Coronaviridae virus in a person, comprising administering to the person an immunologically effective amount of a poliomyelitis vaccine.

In yet another embodiment, provided herein is a method of reducing the severity of one or more symptoms of an infectious disease caused by a Coronaviridae virus in a person, comprising administering to the person an immunologically effective amount of a poliomyelitis vaccine. In one embodiment, the infectious disease is severe acute respiratory syndrome.

In yet another embodiment, provided herein is a method of reducing the severity of one or more symptoms of severe acute respiratory syndrome caused by an infection of a Coronaviridae virus in a person, comprising administering to the person an immunologically effective amount of a poliomyelitis vaccine.

In certain embodiments, the symptom of the severe acute respiratory syndrome is fever or chills, cough, shortness of breath or difficulty breathing, fatigue, muscle or body aches, headache, loss of taste or smell, sore throat, congestion or runny nose, nausea or vomiting, or diarrhea.

In still another embodiment, provided herein is a method of inhibiting replication of a Coronaviridae virus in a person, comprising administering to the person an immunologically effective amount of a poliomyelitis vaccine.

In certain embodiments, the Coronaviridae virus is a Coronavirinae virus. In certain embodiments, the Coronaviridae virus is a Betacoronavirus. In certain embodiments, the Coronaviridae virus is a SARS-CoV-1, SARS-CoV-2, or MERS-CoV. In certain embodiments, the Coronaviridae virus is a SARS-CoV-1. In certain embodiments, the Coronaviridae virus is a SARS-CoV-2. In certain embodiments, the Coronaviridae virus is a MERS-CoV.

In certain embodiments, the poliomyelitis vaccine is an inactivated poliomyelitis vaccine or an attenuated live poliomyelitis vaccine. In certain embodiments, the attenuated live poliomyelitis vaccine is an oral poliomyelitis vaccine (OPV).

In certain embodiments, the poliomyelitis vaccine is a monovalent, divalent, or trivalent poliomyelitis vaccine. In certain embodiments, the poliomyelitis vaccine is a monovalent poliomyelitis vaccine. In certain embodiments, the poliomyelitis vaccine is a divalent poliomyelitis vaccine. In certain embodiments, the poliomyelitis vaccine is a trivalent poliomyelitis vaccine.

In certain embodiments, the poliomyelitis vaccine is an inactivated poliomyelitis vaccine (IPV). In certain embodiments, the poliomyelitis vaccine is an inactivated poliomyelitis vaccine comprising inactivated Type 1 (Mahoney) poliovirus, inactivated Type 2 (MEF-1) poliovirus, inactivated Type 3 (Saukett) poliovirus, or a mixture thereof. In certain embodiments, the poliomyelitis vaccine comprises inactivated Type 1 poliovirus. In certain embodiments, the poliomyelitis vaccine comprises inactivated Type 2 poliovirus. In certain embodiments, the poliomyelitis vaccine comprises inactivated Type 3 poliovirus. In certain embodiments, the poliomyelitis vaccine comprises inactivated Type 1 poliovirus, inactivated Type 2 poliovirus, and inactivated Type 3 poliovirus.

In certain embodiments, the poliomyelitis vaccine is IPOL®. In certain embodiments, the poliomyelitis vaccine is an inactivated poliomyelitis vaccine comprising about 40 D antigen units of inactivated Type 1 poliovirus, about 8 D antigen units of inactivated Type 2 poliovirus, 32 D antigen units of inactivated Type 3 poliovirus, or a mixture thereof. In certain embodiments, the poliomyelitis vaccine is an inactivated poliomyelitis vaccine comprising about 40 D antigen units of inactivated Type 1 poliovirus. In certain embodiments, the poliomyelitis vaccine is an inactivated poliomyelitis vaccine comprising about 8 D antigen units of inactivated Type 2 poliovirus. In certain embodiments, the poliomyelitis vaccine is an inactivated poliomyelitis vaccine comprising 32 D antigen units of inactivated Type 3 poliovirus. In certain embodiments, the poliomyelitis vaccine is an inactivated poliomyelitis vaccine comprising about 40 D antigen units of inactivated Type 1 poliovirus, about 8 D antigen units of inactivated Type 2 poliovirus, and 32 D antigen units of inactivated Type 3 poliovirus.

In certain embodiments, the poliomyelitis vaccine is a monovalent, divalent, or trivalent inactivated poliomyelitis vaccine. In certain embodiments, the poliomyelitis vaccine is a monovalent inactivated poliomyelitis vaccine. In certain embodiments, the poliomyelitis vaccine is a divalent inactivated poliomyelitis vaccine. In certain embodiments, the poliomyelitis vaccine is a trivalent inactivated poliomyelitis vaccine.

In certain embodiments, the poliomyelitis vaccine is an attenuated live poliomyelitis vaccine. In certain embodiments, the poliomyelitis vaccine is an attenuated live poliomyelitis vaccine comprising a Sabin poliovirus. In certain embodiments, the poliomyelitis vaccine is an attenuated live poliomyelitis vaccine comprising Sabin poliovirus I, Sabin poliovirus II, Sabin poliovirus III, or a mixture thereof. In certain embodiments, the poliomyelitis vaccine is an attenuated live poliomyelitis vaccine comprising Sabin poliovirus I (LS-c, 2ab). In certain embodiments, the poliomyelitis vaccine is an attenuated live poliomyelitis vaccine comprising Sabin poliovirus I, having a nucleic acid sequence disclosed in Nomoto et al., Proc. Nat'l. Acad. Sci. U.S.A. 1982, 79, 5793-7, the disclosure of which is incorporated herein by reference in its entirety. In certain embodiments, the poliomyelitis vaccine is an attenuated live poliomyelitis vaccine comprising Sabin poliovirus II (P712, Ch, 2ab). In certain embodiments, the poliomyelitis vaccine is an attenuated live poliomyelitis vaccine comprising Sabin poliovirus III (Leon 12ab). In certain embodiments, the poliomyelitis vaccine is an attenuated live poliomyelitis vaccine comprising Sabin poliovirus I, Sabin poliovirus II, and Sabin poliovirus III.

In certain embodiments, the poliomyelitis vaccine is an attenuated live poliomyelitis vaccine comprising Sabin poliovirus I with a CCID₅₀ of about 10⁶, Sabin poliovirus II with a CCID₅₀ of about 10⁵, Sabin poliovirus III with a CCID₅₀ of about 10⁶, or a mixture thereof. In certain embodiments, the poliomyelitis vaccine is an attenuated live poliomyelitis vaccine comprising Sabin poliovirus I with a CCID₅₀ of about 10⁶. In certain embodiments, the poliomyelitis vaccine is an attenuated live poliomyelitis vaccine comprising Sabin poliovirus II with a CCID₅₀ of about 10⁵. In certain embodiments, the poliomyelitis vaccine is an attenuated live poliomyelitis vaccine comprising Sabin poliovirus III with a CCID₅₀ of about 10⁶. In certain embodiments, the poliomyelitis vaccine is an attenuated live poliomyelitis vaccine comprising Sabin poliovirus I with a CCID₅₀ of about 10⁶, Sabin poliovirus II with a CCID₅₀ of about 10⁵, and Sabin poliovirus III with a CCID₅₀ of about 10⁶.

In certain embodiments, the poliomyelitis vaccine is a monovalent, divalent, or trivalent attenuated live poliomyelitis vaccine. In certain embodiments, the poliomyelitis vaccine is a monovalent attenuated live poliomyelitis vaccine. In certain embodiments, the poliomyelitis vaccine is a divalent attenuated live poliomyelitis vaccine. In certain embodiments, the poliomyelitis vaccine is a trivalent attenuated live poliomyelitis vaccine.

In certain embodiments, the poliomyelitis vaccine is administered orally or parenterally. In certain embodiments, the poliomyelitis vaccine is administered orally. In certain embodiments, the poliomyelitis vaccine is administered parenterally. In certain embodiments, the poliomyelitis vaccine is administered intramuscularly. In certain embodiments, the poliomyelitis vaccine is administered subcutaneously.

In certain embodiments, the inactivated poliomyelitis vaccine is administered orally or parenterally. In certain embodiments, the inactivated poliomyelitis vaccine is administered orally. In certain embodiments, the inactivated poliomyelitis vaccine is administered parenterally. In certain embodiments, the inactivated poliomyelitis vaccine is administered intramuscularly. In certain embodiments, the inactivated poliomyelitis vaccine is administered subcutaneously.

In certain embodiments, the attenuated live poliomyelitis vaccine is administered orally or parenterally. In certain embodiments, the attenuated live poliomyelitis vaccine is administered orally. In certain embodiments, the attenuated live poliomyelitis vaccine is administered parenterally. In certain embodiments, the attenuated live poliomyelitis vaccine is administered intramuscularly. In certain embodiments, the attenuated live poliomyelitis vaccine is administered subcutaneously.

In certain embodiments, the poliomyelitis vaccine is administered from about 1 to about 50 times, from about 1 to about 20 times, from about 1 to about 10, or from about 1 to about 5 times in a lifetime. In certain embodiments, the poliomyelitis vaccine is administered from about 1 to about 50 times in a lifetime. In certain embodiments, the poliomyelitis vaccine is administered from about 1 to about 20 times in a lifetime. In certain embodiments, the poliomyelitis vaccine is administered from about 1 to about 10 in a lifetime. In certain embodiments, the poliomyelitis vaccine is administered from about 1 to about 5 times in a lifetime.

In certain embodiments, the poliomyelitis vaccine is administered once or twice. In certain embodiments, the poliomyelitis vaccine is administered once. In certain embodiments, the poliomyelitis vaccine is administered once. In certain embodiments, the poliomyelitis vaccine is administered once as a booster dose. In certain embodiments, the poliomyelitis vaccine is administered once as a booster dose during a pandemic. In certain embodiments, the poliomyelitis vaccine is administered once as a booster dose during a COVID-19 pandemic.

In certain embodiments, the poliomyelitis vaccine is administered twice within about a year, about 6 months, about 3 months, about 2 months, about 1 months, or about 14 days. In certain embodiments, the poliomyelitis vaccine is administered twice within about a year. In certain embodiments, the poliomyelitis vaccine is administered twice within about 6 months. In certain embodiments, the poliomyelitis vaccine is administered twice within about 3 months. In certain embodiments, the poliomyelitis vaccine is administered twice within about 2 months. In certain embodiments, the poliomyelitis vaccine is administered twice within about 1 months. In certain embodiments, the poliomyelitis vaccine is administered twice within about 14 days.

In certain embodiments, the poliomyelitis vaccine is administered twice with an interval ranging from about 7 days to about 6 months, from about 7 days to about 3 months, from about 7 days to about 2 months, or from about 7 days to about 1 month. In certain embodiments, the poliomyelitis vaccine is administered twice with an interval ranging from about 7 days to about 6 months. In certain embodiments, the poliomyelitis vaccine is administered twice with an interval ranging from about 7 days to about 3 months. In certain embodiments, the poliomyelitis vaccine is administered twice with an interval ranging from about 7 days to about 2 months. In certain embodiments, the poliomyelitis vaccine is administered twice with an interval ranging from about 7 days to about 1 month.

In certain embodiments, the poliomyelitis vaccine is administered twice with the first dose as a priming dose and the second dose as a booster dose. In certain embodiments, the poliomyelitis vaccine is administered twice with both doses as booster doses.

In certain embodiments, the person to be given the poliomyelitis vaccine is unvaccinated against a poliovirus. In certain embodiments, the unvaccinated person receives 1, 2, or 3 doses of the poliomyelitis vaccine. In certain embodiments, the unvaccinated person receives 1 dose of the poliomyelitis vaccine. In certain embodiments, the unvaccinated person receives 2 doses of the poliomyelitis vaccine within about 1 or about two months. In certain embodiments, the unvaccinated person receives 3 doses of the poliomyelitis vaccine within about 3 months or about two years.

In certain embodiments, the person to be given the poliomyelitis vaccine is incompletely vaccinated against a poliovirus. In certain embodiments, the incompletely vaccinated person receives 1 or 2 doses of the poliomyelitis vaccine. In certain embodiments, the incompletely vaccinated person receives 1 dose of the poliomyelitis vaccine. In certain embodiments, the incompletely vaccinated person receives 2 doses of the poliomyelitis vaccine within about 1 or about two months.

In certain embodiments, the person to be given the poliomyelitis vaccine is completely vaccinated. In certain embodiments, the completely vaccinated person receives 1 dose of the poliomyelitis vaccine.

In certain embodiments, the immunologically effective amount of the inactivated poliomyelitis vaccine is ranging from about 1 to about 500, from about 2 to about 200, from about 5 to about 100, or from about 10 to about 100 D antigen units of an inactivated poliovirus. In certain embodiments, the immunologically effective amount of the inactivated poliomyelitis vaccine is ranging from about 1 to about 500 D antigen units of an inactivated poliovirus. In certain embodiments, the immunologically effective amount of the inactivated poliomyelitis vaccine is ranging from about 2 to about 200 D antigen units of an inactivated poliovirus. In certain embodiments, the immunologically effective amount of the inactivated poliomyelitis vaccine is ranging from about 5 to about 100 D antigen units of an inactivated poliovirus. In certain embodiments, the immunologically effective amount of the inactivated poliomyelitis vaccine is ranging from about 10 to about 100 D antigen units of an inactivated poliovirus.

In certain embodiments, the immunologically effective amount of the attenuated live poliomyelitis vaccine has a CCID₅₀ ranging from about 1,000 to about 10¹⁰, from about 10⁴ to about 10⁸, or from about 10⁵ to about 10⁷. In certain embodiments, the immunologically effective amount of the attenuated live poliomyelitis vaccine has a CCID₅₀ ranging from about 1,000 to about 10¹⁰. In certain embodiments, the immunologically effective amount of the attenuated live poliomyelitis vaccine has a CCID₅₀ ranging from about 10⁴ to about 10⁸. In certain embodiments, the immunologically effective amount of the attenuated live poliomyelitis vaccine has a CCID₅₀ ranging from about 10⁵ to about 10⁷.

The disclosure will be further understood by the following non-limiting examples.

### EXAMPLES

### Example 1

### Vaccination Against COVID-19 with Poliomyelitis Vaccine

Four subjects each voluntarily took a booster dose of IPOL® as recommended in the IPOL® Label during the COVID-19 pandemic. Subject 1 (44 year old, female) is a health care professional for more than 20 years. During the COVID-19 pandemic, Subject 1 had been exposed to COVID-19 on many occasions. Subject 1 and her family were planning to travel out of the country. Subject 1 encouraged the members of her family (Subject 2—her husband (47 year old), Subject 3—her sister in her 50s, Subject 4—her father (79 year old), and Subject—her mother (76 year old)) to update all their immunizations prior to the travel. Subjects 1 and 3 to 5 received a booster dose of IPOL® in June 2020 after the COVID19 started to spread in US, but Subject 2 decided not to receive a booster dose.

Subject 2 without receiving a booster dose became sick subsequently and was tested positive for COVID-19 shortly after. Subject 2 had since had unrelenting low-grade fevers (for more than five weeks), some confusion, and chronic weakness. Even though Subjects 1 and 3 to 5 had had frequent contact with Subject 2, none of them became ill.

Subjects 4 and 5, the parents of Subject 1, are elderly and in poor health. Subject 3, a sister of Subject 1, is also in poor health having a history of past stroke and cerebral palsy. All three of them were vaccinated and continued to do well with no signs of illness despite contact with Subject 2.

### Example 2

### Vaccination Against COVID-19 with Poliomyelitis Vaccine

Subject A, a 70 year old generally healthy female, received a booster dose of IPOL® before travelling to Morocco in October 2019. Her husband, Subject B, a 69 year old generally healthy male, received a booster dose of IPOL® per advice of his physician on Oct. 12, 2020. Subjects A and B were married and living together.

On Oct. 14, 2020, their housekeeper had the symptoms of a dry throat, but continued to work in their house. While working in the house, the housekeeper wore a level 3 facemask, a face shield, and gloves. Subject A was in the house during the time when the housekeeper was working. The housekeeper was subsequently tested positive for COVID-19. The housekeeper's husband was also tested and found to be negative at the time.

Five days after her exposure to the housekeeper, Subject A awoke with a sore throat, which started on Oct. 16, 2020 and ran through Oct. 19, 2020. On Oct. 19, 2020, she was tested positive for COVID-19. On Oct. 20, 2020, her symptoms resolved and she was tested again but negative. Subject B, who received the poliomyelitis vaccine booster dose recently, was tested negative for COVID-19 on October 14 and 24, 2020.

In summary, the two elderly subjects (Subjects A and B) were exposed to a COVID-19-positive housekeeper. Subject A, who had a poliomyelitis vaccine booster dose a year ago, developed mild symptoms and tested positive for COVID-19 by a PCT test one day after exposure, but tested negative the following day using the same PCT test. Subject B, who had a poliomyelitis vaccine booster dose recently, never developed symptoms and tested negative during the time.

### Example 3

### Preparation of A Recombinant RDRP

A SARS-CoV-2 RNA dependent RNA polymerase (RDRP) gene encoding region was amplified by PCR from a SARS-CoV-2 cDNA reverse-transcripted from a SARS-CoV-2 viral RNA and cloned into AMERIDX® insect cell expression vector pADX50. After sequencing confirmation, the purified vector was transient transfected into insect cell line Schneider 2 (S2). After induction by an ADX inducer, the RDRP protein expressed was purified by an affinity Ni-NTA column and further purified by DETA SEPHAROSE chromatography.

### Example 4

### Western Blot for Analyzing Human Anti-SARS-CoV-2 RDRP Antibodies

The recombinant poliovirus RDRP and SARS-CoV-2 RDRP (SEQ ID NO: 1) proteins were mixed with an SDS-PAGE loading buffer and separated on a protein gel by electrophoresis. After the proteins on the gel were transferred onto a nitro cellular membrane by electrophoresis, the membrane was blocked with 5% BSA and then treated with a diluted human serum sample to be analyzed for 30 mins. The membrane was washed and incubated with goat anti-human IgG/IgA/IgM conjugated with an HRP enzyme for 30 min. The membrane was washed and treated with an ECL substrate to generate chemiluminescence signals, which were detected and analyzed. The results are summarized in Table 1, where the positive control is an individual who recently received a booster dose of IPOL®.

TABLE 1

| | Before Poliovirus Vaccination | | | After Poliovirus Vaccination | | |
|---|---|---|---|---|---|---|
| Subject | | Poliovirus | SARS-CoV- | | Poliovirus | SARS-CoV- |
| (age, sex) | Poliovirus | RDRP | 2 RDRP | Poliovirus | RDRP | 2 RDRP |
| 12 yr, M | | 1 | — | 3 | 3 | 2.5 |
| 14 yr, F | 3 | 1 | — | 3 | 3 | 2.5 |
| 43 yr, F | — | — | — | 3 | 3 | 2.5 |
| 47 yr, M | — | — | — | 2 | 3 | 1.5 |
| Positive Control | | | | 3 | 3 | 2.5 |

### Example 5

### ELISA Assay for Analyzing Anti-SARS-CoV-2 RDRP Antibodies

The recombinant poliovirus and SARS-CoV-2 RDRP proteins were coated onto an ELISA plate by adding one of the proteins (100 μL) in a coating buffer (100 mM sodium carbonate) to a well. The ELISA plate was incubated at room temperature overnight or 4 hours at 37° C. and then blocked with 5% BSA in a phosphate-buffered saline solution. A diluted human serum or plasma to be analyzed was added to wells and the plate was incubated at room temperature. After washed with an ELISA washing buffer, the plate was incubated with goat anti-human IgG/IgA/IgM conjugated with an HRP enzyme. A TMB substrate was added. The reaction was stopped by adding 2 M sulfuric acid. The plate was read with an ELISA plate reader. The results are summarized in Table 2.

TABLE 2

| Subject | Before Poliovirus | After Poliovirus |
|---|---|---|
| (age, sex) | Vaccination | Vaccination |
| 12 yr, M | 1 | 3 |
| 14 yr, F | 1.5 | 2.5 |
| 43 yr, F | 1 | 3 |
| 47 yr, M | — | 2 |

### Example 6

### Antiviral Activity Against Live SARS-CoV-2

Four serum samples (Samples 1, 2, 3, and 4) were tested for their antiviral activity against a live SARS-CoV-2 (the MEX-BC2/2020 strain). Sample 1 was a serum sample from an individual under 5 year old. Sample 2 was a serum sample from an individual under 5 year old. Sample 3 was a serum from an individual (43 yr, female) receiving a booster dose of IPOL® in June 2020. Sample 4 was a serum from an individual (47 yr, male) prior to the receipt of the booster dose. Two different assay protocols (Protocols 1 and 2) were used to evaluate antiviral activity.

### Antiviral Assay—Protocol 1:

To evaluate antiviral activity against SARS-CoV-2 (MEX-BC2/2020), a CPE-based antiviral assay was performed by infecting Vero E6 cells in the presence or absence of a serum sample. Infection of cells leads to significant cytopathic effect and cell death after 4 days of infection. In this assay, reduction of CPE in the presence of a serum sample was used to determine its antiviral activity. A viability assay was run in parallel for each serum sample.

Vero E6 cells were maintained in DMEM with 10% fetal bovine serum (FBS). The cells were seeded and incubated for 24 h before being pre-incubated with a serum sample. After cell culture was removed from the cells, each serum sample at serial dilutions was added to the cells and incubated for 1 h at 37° C. in a humidified incubator. The cells were then challenged with the viral inoculum resuspended in DMEM with 2% FBS. The amount of viral inoculum was previously titrated to result in a linear inhibitory response by an antiviral with activity against SARS-CoV-2. The cells were incubated in the presence of the virus inoculum and a serum sample for 96 h. The cell viability was then determined using the neutral red uptake assay.

The virus-induced CPE was monitored under the microscope after 3 days of infection and at day 4, the cells were stained with neutral red to determine cell viability. Viable cells incorporate neutral red in their lysosomes. The uptake relies on the ability of live cells to maintain the pH inside the lysosomes lower than in the cytoplasm. This process requires ATP. Inside the lysosome the dye becomes charged and is retained. After a 3 h incubation with neutral red (0.033%), the extra dye was washed and the neutral red taken by lysosomes was then extracted for 15 min with a solution containing 50% ethanol and 1% acetic acid to monitor absorbance at 540 nm.

Each serum sample was evaluated in duplicates using serial 2-fold dilutions. Controls included uninfected cells (“mock-infected”), and infected cells to which only vehicle was added. Full curves of positive plasma from convalescent patient (COV(+)) and GS-441524 were run as positive control inhibitors. GS-441524 is the main metabolite of remdesivir, a broad spectrum antiviral that blocks the RNA polymerase of SARS-CoV-2.

The average absorbance at 540 nm (A540) observed in infected cells (in the presence of vehicle alone) was calculated and then subtracted from all samples to determine the inhibition of the virus induced CPE. Data points were then normalized to the average A540 signal observed in uninfected cells (“mock”) after subtraction of the absorbance signal observed in infected cells. In the neutral red CPE-based assay, uninfected cells remained viable and uptake the dye at higher levels than non-viable cells. In the absence of an antiviral agent, the virus-induced CPE kills infected cells and leads to lower A540 (this value equals 0% inhibition). By contrast, incubation with an antiviral agent (COV(+) or GS-441524) prevents the virus induced CPE and leads absorbance levels similar to those observed in uninfected cells. Full recovery of cell viability in infected cells represent 100% inhibition of virus replication.

### Antiviral Assay—Protocol 2:

To evaluate antiviral activity against SARS-CoV-2 (MEX-BC2/2020), a CPE-based antiviral assay was performed by infecting Vero E6 cells in the presence or absence of a serum sample. Infection of cells leads to significant cytopathic effect and cell death after 4 days of infection. In this assay, reduction of CPE in the presence of a serum sample was used to determine its antiviral activity. A viability assay was run in parallel for each serum sample.

Vero E6 cells were maintained in DMEM with 10% fetal bovine serum (FBS). The cells were seeded and incubated for 24 h before being pre-incubated with virus. After cell culture was removed from the cells, the cells were challenged with the viral inoculum resuspended in DMEM with 2% FBS for 3 h at 37° C. in a humidified incubator. The amount of viral inoculum was previously titrated to result in a linear inhibitory response by an antiviral with activity against SARS-CoV-2. Each serum sample at serial dilutions was then added the infected cells. The cells were incubated in the presence of the virus inoculum and a serum sample for 96 h. The cell viability was then determined using the neutral red uptake assay.

The virus-induced CPE was monitored under the microscope after 3 days of infection and at day 4, the cells were stained with neutral red to determine cell viability. Viable cells incorporate neutral red in their lysosomes. The uptake relies on the ability of live cells to maintain the pH inside the lysosomes lower than in the cytoplasm. This process requires ATP. Inside the lysosome the dye becomes charged and is retained. After a 3 h incubation with neutral red (0.033%), the extra dye was washed and the neutral red taken by lysosomes was then extracted for 15 min with a solution containing 50% ethanol and 1% acetic acid to monitor absorbance at 540 nm.

Each serum sample was evaluated in duplicates using serial 2-fold dilutions. Controls included uninfected cells (“mock-infected”), and infected cells to which only vehicle was added. Full curves of positive plasma from convalescent patient (COV(+)) and GS-441524 were run as positive control inhibitors. GS-441524 is the main metabolite of remdesivir, a broad spectrum antiviral that blocks the RNA polymerase of SARS-CoV-2.

The average absorbance at 540 nm (A540) observed in infected cells (in the presence of vehicle alone) was calculated and then subtracted from all samples to determine the inhibition of the virus induced CPE. Data points were then normalized to the average A540 signal observed in uninfected cells (“mock”) after subtraction of the absorbance signal observed in infected cells. In the neutral red CPE-based assay, uninfected cells remained viable and uptake the dye at higher levels than non-viable cells. In the absence of an antiviral agent, the virus-induced CPE kills infected cells and leads to lower A540 (this value equals 0% inhibition). By contrast, incubation with an antiviral agent (COV(+) or GS-441524) prevents the virus induced CPE and leads absorbance levels similar to those observed in uninfected cells. Full recovery of cell viability in infected cells represent 100% inhibition of virus replication.

### Viability Assay:

Uninfected cells were incubated with the same eight concentrations of a serum sample as used in the antiviral assays. The incubation temperature and duration of the incubation period mirrored the conditions of the prevention of virus-induced CPE assay, and cell viability was evaluated with the same neutral red uptake method as used in the antiviral assays. The extent of viability was monitored by measuring absorbance at 540 nm. When analyzing the data, background levels obtained from wells with no cells were subtracted from all data-points. Absorbance readout values were given as a percentage of the average signal observed in uninfected cells treated with vehicle alone.

### Results:

Antiviral effect was observed for Samples 1 and 3 at 1:8 and 1:32 dilutions. The antiviral effect was stronger when the serum samples were pre-incubated with the Vero E6 cells (Protocol 1) than when the serum samples were added after viral adsorption (Protocol 2). In Protocol 1, Samples 2 and 3 brought the levels of neutral red uptake to about 25% and about 40%, respectively, as compared with uninfected cells. As expected, Samples 2 and 4 did not display significant inhibition of the virus-induced CPE in Protocol 1.

When the serum samples were assessed with Protocol 2, some prevention of the CPE was observed with Sample 4, but neutral red uptake only reached 25-30% of the level observed in uninfected cells. No effect was observed with Samples 1, 2, and 3 when the serum samples were added after viral adsorption.

No cytotoxicity was observed in the viability assay at the concentrations evaluated for each serum sample.

Sequences described herein are provided in the sequence table below.

SEQUENCE TABLE

| SEQ ID | | |
|---|---|---|
| NO: | Description | Amino Acid Sequence |
| 1 | COVID-19 RDRP | SADAQSFLNRVCGVSAARLTPCGTG |
| | | TSTDVVYRAFDIYNDKVAGFAKFLK |
| | | TNCCRFQEKDEDDNLIDSYFVVKRH |
| | | TFSNYQHEETIYNLLKDCPAVAKHD |
| | | FFKFRIDGDMVPHISRQRLTKYTMA |
| | | DLVYALRHFDEGNCDTLKEILVTYN |
| | | CCDDDYFNKKDWYDFVENPDILRVY |
| | | ANLGERVRQALLKTVQFCDAMRNAG |
| | | IVGVLTLDNQDLNGNWYDFGDFIQT |
| | | TPGSGVPVVDSYYSLLMPILTLTRA |
| | | LTAESHVDTDLTKPYIKWDLLKYDF |
| | | TEERLKLFDRYFKYWDQTYHPNCVN |
| | | CLDDRCILHCANFNVLFSTVFPPTS |
| | | FGPLVRKIFVDGVPFVVSTGYHFRE |
| | | LGVVHNQDVNLHSSRLSFKELLVYA |
| | | ADPAMHAASGNLLLDKRTTCFSVAA |
| | | LTNNVAFQTVKPGNFNKDFYDFAVS |
| | | KGFFKEGSSVELKHFFFAQDGNAAI |
| | | SDYDYYRYNLPTMCDIRQLLFVVEV |
| | | VDKYFDCYDGGCINANQVIVNNLDK |
| | | SAGFPFNKWGKARLYYDSMSYEDQD |
| | | ALFAYTKRNVIPTITQMNLKYAISA |
| | | KNRARTVAGVSICSTMTNRQFHQKL |
| | | LKSIAATRGATVVIGTSKFYGGWHN |
| | | MLKTVYSDVENPHLMGWDYPKCDRA |
| | | MPNMLRIMASLVLARKHTTCCSLSH |
| | | RFYRLANECAQVLSEMVMCGGSLYV |
| | | KPGGTSSGDATTAYANSVFNICQAV |
| | | TANVNALLSTDGNKIADKYVRNLQH |
| | | RLYECLYRNRDVDTDFVNEFYAYLR |
| | | KHFSMMILSDDAVVCFNSTYASQGL |
| | | VASIKNFKSVLYYQNNVFMSEAKCW |
| | | TETDLTKGPHEFCSQHTMLVKQGDD |
| | | YVYLPYPDPSRILGAGCFVDDIVKT |
| | | DGTLMIERFVSLAIDAYPLTKHPNQ |
| | | EYADVFHLYLQYIRKLHDELTGHML |
| | | DMYSVMLTNDNTSRYWEPEFYEAMY |
| | | TPHTVLQ |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1-3. (canceled)

4. A method of reducing the severity of one or more symptoms of an infectious disease caused by a Coronaviridae virus in a person, comprising administering to the person an immunologically effective amount of a poliomyelitis vaccine.

5. The method of claim 4, wherein the infectious disease is severe acute respiratory syndrome.

6. A method of inhibiting replication of a Coronaviridae virus in a person, comprising administering to the person an immunologically effective amount of a poliomyelitis vaccine.

7. The method of claim 4, wherein the Coronaviridae virus is a Coronavirinae virus.

8-9. (canceled)

10. The method of claim 4, wherein the Coronaviridae virus is a severe acute respiratory syndrome coronavirus-2.

11. (canceled)

12. The method of claim 4, wherein the poliomyelitis vaccine is a monovalent, divalent, or trivalent poliomyelitis vaccine.

13. The method of claim 4, wherein the poliomyelitis vaccine is an inactivated poliomyelitis vaccine.

14. The method of claim 13, wherein the inactivated poliomyelitis vaccine comprises inactivated Type 1 (Mahoney) poliovirus, inactivated Type 2 (MEF-1) poliovirus, inactivated Type 3 (Saukett) poliovirus, or a mixture thereof.

15-17. (canceled)

18. The method of claim 13, wherein the inactivated poliomyelitis vaccine comprises inactivated Type 1 poliovirus, inactivated Type 2 poliovirus, and inactivated Type 3 poliovirus.

19. The method of claim 13, wherein the immunologically effective amount is ranging from about 1 to about 500 D antigen units of an inactivated poliovirus.

20. The method of claim 13, wherein the inactivated poliomyelitis vaccine comprises about 40 D antigen units of inactivated Type 1 poliovirus, about 8 D antigen units of inactivated Type 2 poliovirus, and 32 D antigen units of inactivated Type 3 poliovirus.

21. The method of claim 4, wherein the poliomyelitis vaccine is an attenuated live poliomyelitis vaccine.

22. The method of claim 21, wherein the attenuated live poliomyelitis vaccine comprises Sabin poliovirus I, Sabin poliovirus II, Sabin poliovirus III, or a mixture thereof.

23-24. (canceled)

25. The method of claim 21, wherein the attenuated live poliomyelitis vaccine comprises Sabin poliovirus I, Sabin poliovirus II, and Sabin poliovirus III.

26. The method of claim 21, wherein the immunologically effective amount of the attenuated live poliomyelitis vaccine has a CCID₅₀ ranging from about 1,000 to about 10¹⁰, from about 10⁴ to about 10⁸, or from about 10⁵ to about 10⁷.

27. The method of claim 21, wherein the attenuated live poliomyelitis vaccine comprises Sabin poliovirus I with a CCID₅₀ of about 10⁶, Sabin poliovirus II with a CCID₅₀ of about 10⁵, and Sabin poliovirus III with a CCID₅₀ of about 10⁶.

28. (canceled)

29. The method of claim 21, wherein the poliomyelitis vaccine is administered orally.

30. The method of claim 13, wherein the poliomyelitis vaccine is administered parenterally.

31. The method of claim 30, wherein the poliomyelitis vaccine is administered intramuscularly or subcutaneously.

32-37. (canceled)


SEQUENCE LISTING (1 sequence)

SEQ ID NO: 1 - protein, 932 aa, Homo sapiens
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ